(19) United States
(12) Patent Application Publication
(10) Pub. No.: US 2017/0113979 A1
(43) Pub. Date: Apr. 27, 2017

(54) RETAINING CIRCULATION OF IONIC LIQUID DURING AN EMERGENCY OR PROCESS UPSET OF IONIC LIQUID ALKYLATION PROCESS

(71) Applicant: UOP LLC, Des Plaines, IL (US)

(72) Inventors: David Scott Lafyatis, Schaumburg, IL (US); Douglas Andrew Nafis, Mt. Prospect, IL (US); Robert James Schmidt, Barrington, IL (US); Matthew Lippmann, Chicago, IL (US); Eric Leeton, Corpus Christi, TX (US); Kurt Detrick, Glen Ellyn, IL (US)

(21) Appl. No.: 14/919,384

(22) Filed: Oct. 21, 2015

(51) Int. Cl. C07C 2/58

(52) U.S. Cl. CPC C07C 2/58; C07C 2531/02

(57) ABSTRACT

An alkylation process system uses an ionic liquid as a catalyst which undergoes an interruption in normal operating condition. A method of maintaining the alkylation process system during the interruption of the normal operating condition requires maintaining a circulation of the ionic liquid through the alkylation process system without interruption.

### CROSS-REFERENCE TO RELATED APPLICATIONS

N/A

### FEDERALLY SPONSORED RESEARCH OR DEVELOPMENT

N/A

### TECHNICAL FIELD

The invention relates generally to processes using an ionic liquid as a catalyst; more particularly, the invention relates to a method of maintaining circulation of an ionic liquid within the process during a during an interruption of the process while optionally not requiring the netting of any product, so that when normal conditions have been restored, the ionic liquid process will be able to rapidly return to normal operation.

### BACKGROUND OF THE INVENTION

Alkylation processes using acidic ionic liquids as the catalyst, such as the alkylation of isobutane with butene, or other C3 and C5 olefins, to generate high octane alkylate, have been demonstrated to be viable alternatives to alkylation with acids such as HF or H₂SO₄. Although there is a preponderance of information available regarding steady-state operation of such a process, one problem associated with such a process is the limited information regarding methods of operating the ionic liquid alkylation process during non-standard operation outside of specification and during other upset conditions.

The present invention is provided to solve the problems discussed above and other problems, and to provide advantages and aspects not disclosed for prior processes of this type. Recovery from such process upsets, which are virtually unavoidable in plant operation, can be a key economic driver for the process. A full discussion of the features and advantages of the present invention is deferred to the following detailed description, which proceeds with reference to the accompanying drawings.

### SUMMARY OF THE INVENTION

One aspect of the present invention is directed to a method of maintaining an alkylation process system that uses an ionic liquid (IL) as a catalyst, during an interruption of a normal operating condition. The method comprises the step of maintaining a circulation of the ionic liquid through the alkylation process system without interruption. Such continued circulation is considered critical to avoid well-known problems such as IL settling, crystallization, solidifying, or other related issues.

The first aspect of the invention may include one or more of the following features, alone or in any reasonable combination. The interruption in the normal operating condition may be caused by a loss of one of a plurality of feeds to a reactor in the alkylation process system. The plurality of feeds may comprise an olefin feed and an isoparaffin feed. The interruption in the normal operating condition may be caused by a loss the olefin feed wherein the isoparaffin feed (frequently isobutane) is continually introduced into the alkylation process system during the interruption in the normal operating condition. A loss of the olefin feed reactant may cause a cessation of an alkylate product formation wherein the ionic liquid catalyst remains in circulation at a predetermined flow rate, a level in a fractionator is monitored and controlled, and an alkylate product yield from the alkylation process system is substantially diminished or stopped entirely. The interruption in the normal operating condition may be caused by a loss the isoparaffin feed wherein introduction of the olefin feed into the alkylation process system is intentionally interrupted during the interruption in the normal operating condition. A loss of the olefin feed reactant may cause a cessation of an alkylate product formation, the ionic liquid catalyst remains in circulation at a predetermined flow rate, a level in a fractionator is monitored and controlled, and an alkylate product yield from the alkylation process system is substantially diminished or stopped entirely. The interruption in the normal operating condition may be caused by a loss of an ability to deliver a product stream from a reactor in the alkylation process system to a designed destination. The interruption in the normal operating condition may be caused by a loss of an ability to purge reject streams comprising one or more of a contaminate reject stream and a non-condensable reject stream. The interruption in the normal operating condition may be caused by a loss of a chloride make-up. The interruption in the normal operating condition may be caused by a loss of an ability to provide adequate regenerated ionic liquid in the alkylation process system. The loss of the ability to provide adequate regenerated ionic liquid may be caused by an inability to feed a new volume of fresh ionic liquid into the alkylation process system. The loss of the ability to regenerate the ionic liquid may be caused by a loss of the ability to purge spent ionic liquid from the alkylation process system. The step of maintaining the circulation of the ionic liquid through the alkylation process system without interruption may include maintaining mechanical operation of equipment necessary to ensure proper ionic liquid droplet size and size distribution within the alkylation process within the alkylation process system. The step of maintaining the circulation of the ionic liquid through the alkylation process system without interruption may include distribution of the circulation of the ionic liquid through the alkylation process system. The method may further comprise decreasing a volume flow of a fractionator overhead stream. The method may further comprise operating an ionic liquid regeneration section of the alkylation process system at a predetermined rate. The method may further comprise maintaining at least one of a plurality of secondary processes and utility systems in the alkylation process system at a predetermined operating level wherein the at least one of the plurality of secondary processes and utility systems is chosen from the group consisting of: pump flushes, seal flushes, seal barrier fluid streams, packing purges and flushes, annular purges and flushes, instrument purges and flushes, cooling water systems, cooling water chiller systems, vent and flare gas scrubbing systems, and liquid waste and/or process fluid disposal systems. The method may further comprise restoring ionic liquid flow through the alkylation process system to an operating level; and restoring a chloride balance in the alkylation process system to an operating level by performing at least one of a group of steps consisting of: venting of chlorides from the alkylation process system; introducing anhydrous hydrogen chloride to the alkylation process system; and introducing a chloride containing material to the alkylation process system, wherein the restoring steps are performed prior to the step of reintroducing one of an isobutane feed or an olefin feed to a reactor in the alkylation process system.

Other features and advantages of the invention will be apparent from the following specification taken in conjunction with the following drawings.

### BRIEF DESCRIPTION OF THE DRAWINGS

To understand the present invention, it will now be described by way of example, with reference to the accompanying drawing in which:

FIG. 1 is a basic schematic representation of an alkylation process to which the method of the present invention may be applied.

### DETAILED DESCRIPTION

While this invention is susceptible of embodiments in many different forms, there is shown in the drawing and will herein be described in detail preferred embodiments of the invention with the understanding that the present disclosure is to be considered as an exemplification of the principles of the invention and is not intended to limit the broad aspect of the invention to the embodiments illustrated.

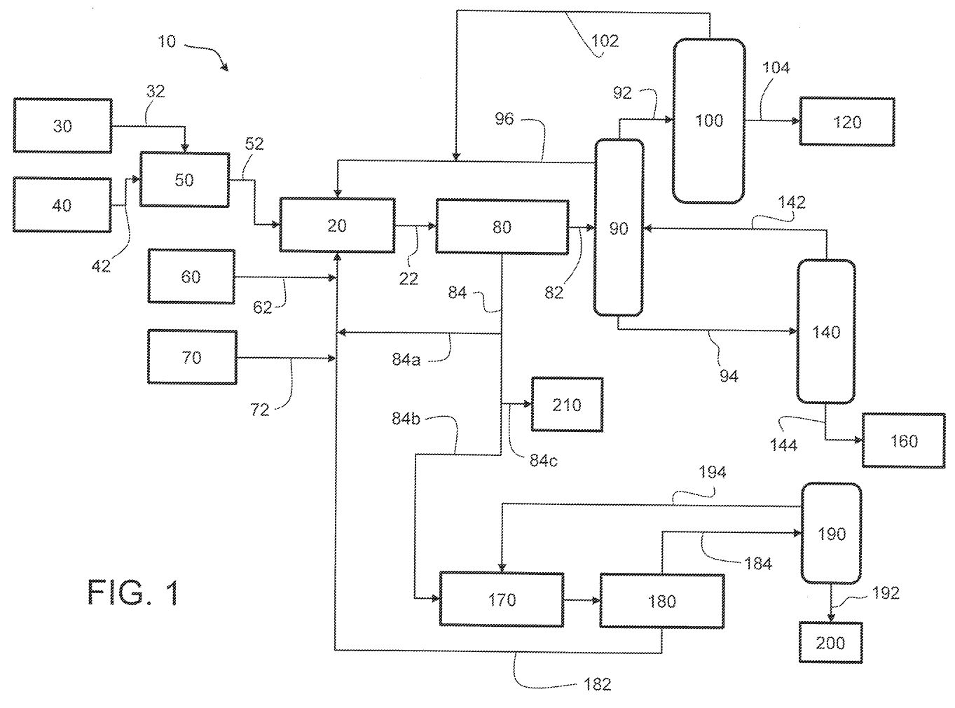

An alkylation process system 10 is shown in FIG. 1. The system 10 includes one or more reactors 20. The reactors 20 operate on an ionic liquid catalyst and convert olefin and other feeds, generally outputting a stream 22 comprising alkylate, unreacted isoparaffin, chloride, and ionic liquid. The ionic liquid typically represents about 1-20% of a liquid volume in the reactors 20 with the remaining volume being predominately hydrocarbons with a small amount of chloride.

The reactors 20 are in upstream fluid communication with a source of an olefin 30, which provides an olefin feed 32, such as butene or isobutene, and a source of isoparaffin 40, which provides an isoparaffin feed 42 such as isobutane, to the reactors 20. These sources 30, 40 provide feed streams to the reactor 20 via a treater 50. The treater 50 removes moisture and contaminates, such as sulfur, from the feeds 32, 42. The treater 50 preferably delivers a dry, nearly pure hydrocarbon feed 52 comprising the dried and decontaminated olefin and isoparaffin feeds 32, 42 to the reactors 20.

A source of chloride 60, typically an organic chloride or HCl delivers a stream of make-up chloride 62 to the reactors 20 to offset system loses of the chloride. This may be added in at Line 62 as shown in this diagram, or alternatively may be added at stream 94. A source of ionic liquid 70 passes a stream of make-up ionic liquid 72 to the reactors 20 to offset system loses of the catalyst if present.

A reactor effluent separator 80 is in downstream fluid communication with the reactors 20. A rough separation process is performed within the separator 80. It receives the stream 22 from the reactors 20 and passes a hydrocarbon stream 82 and a bottom stream 84. The hydrocarbon stream 82 comprises alkylate, unreacted isoparaffin, and chloride. The bottom stream 84 comprises ionic liquid, conjunct polymer and may also contain chloride.

The hydrocarbon stream 82 is delivered to a downstream fractionator or series of fractionators 90. The fractionator 90 outputs a chloride containing stream 92, a first hydrocarbon stream 94, and a second hydrocarbon stream 96. The chloride containing stream 92 comprises anhydrous HCl and some unreacted isoparaffin. This stream 92 passes to a chloride stripper 100 which outputs a stream of anhydrous HCl 102 eventually back to the reactors 20 and a hydrocarbon stream 104 which is purged via stream 120 from the system 10. The C4 or higher hydrocarbons may be sent to an additional process unit for isomerization and eventual return back to the alkylation unit.

The first hydrocarbon stream 94 from fractionator 90 passes to an alkylate stripper 140 where a stream comprising chlorides and butanes 142 is passed back to fractionator 90, and an alkylate stream 144 passes to final product 160 where it is taken from the system 10.

The second hydrocarbon stream 96 comprises isoparaffin. This stream 96 is circulated back to the reactors 20.

A portion 84a of bottom stream 84 taken from the reactor effluent separator 80 is transferred back to the reactors 20. A second portion 84b passes to a regeneration section 170. Here, this section 170 restores spent or used ionic liquid catalyst for alkylation and removes accumulated conjunct polymer. A regeneration section stripper 180 receives the bottom stream 84b from separator 80 and drops a regenerated ionic liquid stream 182 as a bottom stream which is transferred back to the reactors 20. A hydrocarbon stream 184 is transferred to column 190 where a conjunct polymer stream 192 drops from the bottom and is pulled or removed from the system 10 at operation 200 and an isobutane stream 194 recirculates to the regeneration section 170.

A third portion 84c of the bottom stream 84 taken from the reactor effluent separator 80 comprises mostly spent ionic liquid which has lost activity over time. This portion 84c may be dragged from the system 10 at process 210.

When a system upset occurs, such as loss of olefin feed 32 or other feed stocks, it is critical that the ionic liquid does not become or remain stagnant. Thus, there is a need for a method to ensure that the ionic liquid catalyst remains sufficiently mixed and flowing in the system 10.

The present invention is directed to a method of maintaining an alkylation system 10 that uses an ionic liquid as a catalyst, during an interruption of a normal operating condition. The invention described herein provides a process methodology to retain circulation and movement of the ionic liquid through a system while netting no or a substantially reduced amount of any alkylate product. When normal conditions have been restored, the system 10 will be able to rapidly return to normal operation.

Several circumstances may cause the interruption of the normal operating condition. For example, the interruption can be caused by a loss of one of a plurality of feeds to a reactor 20 in the alkylation process system. As explained above, the plurality of feeds includes an olefin feed 32 and an isoparaffin feed 42, e.g. an isobutane. Loss as used herein refers to at least a 50% decrease in volume flow relative to standard operation; a nearly complete loss as used herein refers to a 90% or more decrease in volume flow relative to standard operation; and a complete loss refers to a 100% decrease in volume flow relative to standard operation.

In the event of a loss of the olefin feed 32, the isoparaffin feed 42 is continually introduced into the alkylation process system 10 during the interruption. This type of loss causes a cessation or a substantial reduction of an alkylate product formation, and the ionic liquid catalyst remains in circulation through the reactor 20 and the separator 80 at a predetermined flow rate. A level in the fractionator 90 and alkylate stripper 140 is monitored and controlled, and an alkylate product yield 160 from the alkylation system 10 is stopped or substantially reduced.

To stabilize fractionator operation, the second hydrocarbon stream 96 comprising alkylate must continue to be recirculated through the fractionator 90 and alkylate stripper 140. During the interruption, pressures and temperatures in the fractionator 90 and alkylate stripper 140, surge drums, receivers, accumulators, and other similar vessels must be controlled and maintained at design values or within some other specified parameters.

The isoparaffin-comprising stream 96 recirculated from the fractionator 90 to the reactors 20 may be bypassed, partially bypassed, or flowed through the reactors 20 at design values or at some other specified rate. The streams 92, 102, 142 may be stopped, operated at design values or some other specified value, or may be yielded from the unit as specified.

The loss of isoparaffin 42 requires that the olefin feed 32 is also substantially reduced or stopped.

Additionally, the method of the present invention is performed when a loss in an ability to deliver the product stream 22 from the reactor 20 to a designed destination occurs, e.g. the reactor effluent separator 80, or when a loss in an ability to purge reject comprising one or more of a contaminate reject stream and a non-condensable reject stream occurs.

Other circumstances causing interruption during which circulation of the ionic liquid would be maintained include a loss of the chloride make-up stream 62 into the system 10 and/or reactors 20, a loss of an ability to regenerate the ionic liquid and deliver the stream of regenerated ionic liquid 182 back to the reactors 20, a loss of an ability to deliver the ionic liquid make-up stream 72 to the system and/or reactors 20. The loss of the ability to deliver the regenerated ionic liquid stream 182 to the reactors 20 may be caused by an inability to purge spent ionic liquid from the alkylation system 10.

There are key considerations to preventing the ionic liquid from settling in the various processes that make up the system 10 and to prevent fouling of equipment and solidification of the ionic liquid in the system 10. For example, during the interruption mixers, agitators, and other mechanical means of controlling ionic liquid droplet size and distribution in the reactor 20 and/or product separation section must remain in operation.

During the interruption, the ionic liquid regeneration section 170 and associated equipment may be stopped but is typically left operating at design or some other specified rate. This may include decreasing a volume flow of a fractionator overhead (e.g. streams 102, 142, 184, and/or 194), side-cut (e.g. stream 96), or bottoms stream (e.g. stream 84 and/or 182).

Given the unique nature and characteristics of the ionic liquid, secondary process and utility systems must be kept in operation at design or some other specified value. These streams may be, but are not limited to, the following: pump flushes, seal flushes, seal barrier fluid streams, packing purges and flushes, annular purges and flushes, instrument purges and flushes; cooling water systems; cooling water chiller systems; vent and flare gas scrubbing systems; liquid waste and process fluid disposal systems; chloride treaters; etc.

Given the unique nature and characteristics of ionic liquids and to ensure the continued safe operation of the ionic liquid process, safety systems and equipment such as, but not limited to: interlocks; relief valves, emergency interlock systems, etc. must remain in operation.

Given the unique nature and characteristics of ionic liquids it may be necessary to collect and analyze representative samples of unit process streams utilizing but not limited to means such as online analyzers, established laboratory methods or some other specified analytical methods.

Given the unique physical properties of ionic liquid, personal protective equipment (PPE) such as, but not limited to, chemical resistant gloves, chemical resistant trousers or pants, chemical resistant jackets, coats or overalls, hoods or other similar head covering, chemical resistant footwear and respiratory protection such as, but not limited to, cartridge respirators, positive pressure full face respirators, hose line or self-contained breathing apparatus (SCBA) must be employed as specified.

Prior to reintroduction of the olefin and isoparaffin feeds 32, 42, the ionic liquid catalyst activity and chloride balance in the system 10 may have to be restored to design values or some other specified concentration by the venting of chlorides from the system 10 or by the addition of anhydrous hydrogen chloride or some other suitable chloride containing material. Conjunct Polymer (CP), accumulated contaminants and ionic liquid catalyst activity may have to be restored to design or some other specified value by operation of the regeneration system 170 prior to the reintroduction of the olefin and isoparaffin feeds 32, 42.

### SPECIFIC EMBODIMENTS

While the following is described in conjunction with specific embodiments, it will be understood that this description is intended to illustrate and not limit the scope of the preceding description and the appended claims.

A first embodiment of the invention is a method of maintaining an alkylation system that uses an ionic liquid as a catalyst, during an interruption of a normal operating condition by maintaining a circulation of the ionic liquid through the alkylation system without interruption. An embodiment of the invention is one, any or all of prior embodiments in this paragraph up through the first embodiment in this paragraph wherein the interruption in the normal operating condition is caused by a loss of one of a plurality of feeds to a reactor in the alkylation system. An embodiment of the invention is one, any or all of prior embodiments in this paragraph up through the first embodiment in this paragraph wherein the plurality of feeds comprises an olefin feed and an isoparaffin feed. An embodiment of the invention is one, any or all of prior embodiments in this paragraph up through the first embodiment in this paragraph wherein the interruption in the normal operating condition is caused by a loss the olefin feed wherein the isoparaffin feed is continually introduced into the alkylation system during the interruption in the normal operating condition. An embodiment of the invention is one, any or all of prior embodiments in this paragraph up through the first embodiment in this paragraph wherein a loss of the olefin feed reactant causes a substantial reduction or cessation of an alkylate product formation, the ionic liquid catalyst remains in circulation at a predetermined flow rate, a level in a fractionator is monitored and controlled, and an alkylate product yield from the alkylation process system is substantially reduced or stopped. An embodiment of the invention is one, any or all of prior embodiments in this paragraph up through the first embodiment in this paragraph wherein the interruption in the normal operating condition is caused by a loss the isoparaffin feed to a reactor wherein introduction of the olefin feed into the alkylation system is intentionally interrupted during the interruption in the normal operating condition. An embodiment of the invention is one, any or all of prior embodiments in this paragraph up through the first embodiment in this paragraph wherein a loss of the olefin feed reactant causes a substantial reduction or cessation of an alkylate product formation, the ionic liquid catalyst remains in circulation through a reactor 20 and a separator 80 at a predetermined flow rate, a level in a fractionator is monitored and controlled, and an alkylate product yield from the alkylation system is substantially reduced or stopped. An embodiment of the invention is one, any or all of prior embodiments in this paragraph up through the first embodiment in this paragraph wherein the interruption in the normal operating condition is caused by a loss of an ability to deliver a product stream from a reactor in the alkylation system to a designed destination. An embodiment of the invention is one, any or all of prior embodiments in this paragraph up through the first embodiment in this paragraph wherein the interruption in the normal operating condition is caused by a loss of an ability to purge reject streams comprising one or more of a contaminant reject stream and a non-condensable reject stream. An embodiment of the invention is one, any or all of prior embodiments in this paragraph up through the first embodiment in this paragraph wherein the interruption in the normal operating condition is caused by a loss of a chloride make-up stream. An embodiment of the invention is one, any or all of prior embodiments in this paragraph up through the first embodiment in this paragraph wherein the interruption in the normal operating condition is caused by a loss of an ability to regenerate the ionic liquid in the alkylation system. An embodiment of the invention is one, any or all of prior embodiments in this paragraph up through the first embodiment in this paragraph wherein the interruption in the normal operating condition is caused by a loss of an ability to feed a new volume of the ionic liquid into the alkylation process system. An embodiment of the invention is one, any or all of prior embodiments in this paragraph up through the first embodiment in this paragraph wherein the loss of the ability to regenerate the ionic liquid is caused by a loss of the ability to purge spent ionic liquid from the alkylation system. An embodiment of the invention is one, any or all of prior embodiments in this paragraph up through the first embodiment in this paragraph wherein maintaining the circulation of the ionic liquid through the alkylation system without interruption includes maintaining mechanical operation of an ionic liquid droplet size controller within the alkylation system. An embodiment of the invention is one, any or all of prior embodiments in this paragraph up through the first embodiment in this paragraph wherein maintaining the circulation of the ionic liquid through the alkylation system without interruption includes distribution of the circulation of the ionic liquid through the alkylation system. An embodiment of the invention is one, any or all of prior embodiments in this paragraph up through the first embodiment in this paragraph further comprising the step of decreasing a volume flow of a fractionator overhead, side-cut, or bottoms stream. An embodiment of the invention is one, any or all of prior embodiments in this paragraph up through the first embodiment in this paragraph further comprising the step of operating an ionic liquid regeneration section of the alkylation system at a predetermined rate. An embodiment of the invention is one, any or all of prior embodiments in this paragraph up through the first embodiment in this paragraph further comprising the step maintaining at least one of a plurality of secondary processes and utility systems in the alkylation system at a predetermined operating level wherein the at least one of the plurality of secondary processes and utility systems is chosen from the group consisting of pump flushes, seal flushes, seal barrier fluid streams, packing purges and flushes, annular purges and flushes, instrument purges and flushes, cooling water systems, cooling water chiller systems, vent and flare gas scrubbing systems, liquid waste and process fluid disposal systems, and chloride treaters. An embodiment of the invention is one, any or all of prior embodiments in this paragraph up through the first embodiment in this paragraph further comprising the steps of restoring ionic liquid flow through the alkylation system to an operating level; and restoring a chloride balance in the alkylation system to an operating level by performing at least one of a group of steps consisting of venting of chlorides from the alkylation system; introducing an anhydrous hydrogen chloride to the alkylation system; and introducing a chloride containing material to the alkylation system, wherein the restoring steps are performed prior to the step of reintroducing one of an isoparaffin feed or an olefin feed to a reactor in the alkylation system. An embodiment of the invention is one, any or all of prior embodiments in this paragraph up through the first embodiment in this paragraph wherein the interruption in the normal operating condition is caused by a nearly complete loss or a complete loss of one of a plurality of feeds to a reactor in the alkylation system.

A second embodiment of the invention is a method of maintaining an alkylation system that uses an ionic liquid as a catalyst, during an interruption of a normal operating condition by maintaining a first flow path of the ionic liquid through the alkylation system. An embodiment of the invention is one, any or all of prior embodiments in this paragraph up through the first embodiment in this paragraph wherein the first flow path comprises ionic liquid flow to and through a reactor from the reactor to and through a reactor effluent separator and back to the reactor. An embodiment of the invention is one, any or all of prior embodiments in this paragraph up through the first embodiment in this paragraph wherein the first flow path further comprises ionic liquid flow from the reactor effluent separator to and through an ionic liquid regeneration section, from the ionic liquid regeneration section to and through an ionic liquid regeneration stripper and from the ionic liquid regeneration stripper back to the reactor. An embodiment of the invention is one, any or all of prior embodiments in this paragraph up through the first embodiment in this paragraph further comprising maintaining a second flow path wherein the second flow path comprises isobutane flow to and through a reactor, from the reactor to and through a reactor effluent separator, from the reactor effluent separator to and through a fractionator, and from the fractionator back to the reactor. An embodiment of the invention is one, any or all of prior embodiments in this paragraph up through the first embodiment in this paragraph wherein the second flow path further comprises isobutane flow from the fractionator to and through a chloride stripper, and from the chloride stripper back to the reactor.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

While the specific embodiments have been illustrated and described, numerous modifications come to mind without significantly departing from the spirit of the invention, and the scope of protection is only limited by the scope of the accompanying Claims.

What is claimed is:

1. A method of maintaining an alkylation system that uses an ionic liquid as a catalyst, during an interruption of a normal operating condition by maintaining a circulation of the ionic liquid through the alkylation system without interruption.

2. The method of claim 1 wherein the interruption in the normal operating condition is caused by a loss of one of a plurality of feeds to a reactor in the alkylation system.

3. The method of claim 2 wherein the plurality of feeds comprises an olefin feed and an isoparaffin feed.

4. The method of claim 3 wherein the interruption in the normal operating condition is caused by a loss the olefin feed wherein the isoparaffin feed is continually introduced into the alkylation system during the interruption in the normal operating condition.

5. The method of claim 4 wherein a loss of the olefin feed reactant causes a substantial reduction or cessation of an alkylate product formation, the ionic liquid catalyst remains in circulation at a predetermined flow rate, a level in a fractionator is monitored and controlled, and an alkylate product yield from the alkylation process system is substantially reduced.

6. The method of claim 3 wherein the interruption in the normal operating condition is caused by a loss the isoparaffin feed to a reactor wherein introduction of the olefin feed into the alkylation system is intentionally interrupted during the interruption in the normal operating condition.

7. The method of claim 6 wherein a loss of the olefin feed reactant causes a substantial reduction or cessation of an alkylate product formation, the ionic liquid catalyst remains in circulation at a predetermined flow rate, a level in a fractionator is monitored and controlled, and an alkylate product yield from the alkylation system is substantially reduced.

8. The method of claim 1 wherein the interruption in the normal operating condition is caused by at least one of a group of conditions consisting of:
a loss of an ability to deliver a product stream from a reactor in the alkylation system to a designed destination, a loss of an ability to purge reject streams comprising one or more of a contaminant reject stream and a non-condensable reject stream, a loss of a chloride make-up stream, a loss of an ability to regenerate the ionic liquid in the alkylation system, and a loss of an ability to feed a new volume of the ionic liquid into the alkylation process system.

9. The method of claim 8 wherein the loss of the ability to regenerate the ionic liquid is caused by a loss of the ability to purge spent ionic liquid from the alkylation system.

10. The method of claim 1 wherein maintaining the circulation of the ionic liquid through the alkylation system without interruption includes maintaining mechanical operation of a means to control ionic liquid droplet size within the alkylation system.

11. The method of claim 1 wherein maintaining the circulation of the ionic liquid through the alkylation system without interruption includes distribution of the circulation of the ionic liquid through the alkylation system.

12. The method of claim 1 further comprising the step of decreasing a volume flow of a fractionator overhead, side-cut, or bottoms stream.

13. The method of claim 1 further comprising the step maintaining at least one of a plurality of secondary processes and utility systems in the alkylation system at a predetermined operating level wherein the at least one of the plurality of secondary processes and utility systems is chosen from the group consisting of: pump flushes, seal flushes, seal barrier fluid streams, packing purges and flushes, annular purges and flushes, instrument purges and flushes, cooling water systems, cooling water chiller systems, vent and flare gas scrubbing systems, liquid waste and process fluid disposal systems, and chloride treaters.

14. The method of claim 13 further comprising the steps of:
restoring ionic liquid flow through the alkylation system to an operating level; and
restoring a chloride balance in the alkylation system to an operating level by performing at least one of a group of steps consisting of:
venting of chlorides from the alkylation system;
introducing an anhydrous hydrogen chloride to the alkylation system; and
introducing a chloride containing material to the alkylation system,
wherein the restoring steps are performed prior to the step of reintroducing one of an isoparaffin feed or an olefin feed to a reactor in the alkylation system.

15. The method of claim 1 wherein the interruption in the normal operating condition is caused by a nearly complete loss or a complete loss of one of a plurality of feeds to a reactor in the alkylation system.

16. A method of maintaining an alkylation system that uses an ionic liquid as a catalyst, during an interruption of a normal operating condition by maintaining a first flow path of the ionic liquid through the alkylation system.

17. The method of claim 16 wherein the first flow path comprises ionic liquid flow to and through a reactor, from the reactor to and through a reactor effluent separator, and back to the reactor.

18. The method of claim 17 wherein the first flow path further comprises ionic liquid flow from the reactor effluent separator to and through an ionic liquid regeneration section, from the ionic liquid regeneration section to and through an ionic liquid regeneration stripper, and from the ionic liquid regeneration stripper back to the reactor.

19. The method of claim 16 further comprising maintaining a second flow path wherein the second flow path comprises isobutane flow to and through a reactor, from the reactor to and through a reactor effluent separator, from the reactor effluent separator to and through a fractionator, and from the fractionator back to the reactor.

20. The method of claim 19 wherein the second flow path further comprises isobutane flow from the fractionator to and through a chloride stripper, and from the chloride stripper back to the reactor.
